(19) United States
(12) Patent Application Publication
(10) Pub. No.: US 2022/0332974 A1
(43) Pub. Date: Oct. 20, 2022

(54) POLYSILOXANES AND FLUOROSILANES ON INSULATION ELEMENTS

(71) Applicants: Kester Julian Batchelor, Mound, MN (US); Teo Heng Jimmy Yang, Heath (GB)

(72) Inventors: Kester Julian Batchelor, Mound, MN (US); Teo Heng Jimmy Yang, Heath (GB)

(21) Appl. No.: 17/659,637

(22) Filed: Apr. 18, 2022

Related U.S. Application Data
(60) Provisional application No. 63/175,922, filed on Apr. 16, 2021.

(51) Int. Cl. C09D 183/04; A61B 18/14

(52) U.S. Cl. CPC C09D 183/04; A61B 18/14; A61B 2018/00601

(57) ABSTRACT

Various embodiments disclosed relate to a non-stick layer for insulative elements on electrosurgical cutting tools. The present disclosure includes systems, devices, and methods of making and using a non-stick layer on insulative element. The non-stick layer can include coatings, surface structures, or combinations thereof.

### CROSS-REFERENCE TO RELATED APPLICATION

This application claims the benefit of priority to U.S. Provisional Patent Application Serial No. 63/175,922 filed Apr. 16, 2021, the contents of which are hereby incorporated by reference in their entirety.

### TECHNICAL FIELD

This document pertains generally, but not by way of limitation, to electrosurgical devices that can be used for various surgical procedures.

### BACKGROUND

Electrosurgery uses the application of a high frequency alternating polarity electrical current, such as a radio frequency (RF) electrical current, to cut, coagulate, desiccate, or fulgurate tissue. The high frequency alternating current (AC) can be converted to heat by resistance as it passes through tissue. The result of heat buildup within the tissue can be used to cause tissue thermal damage, resulting in effects such as cutting or cautery of tissue. Electrosurgery can allow for high precision cutting in surgery with low blood loss.

The application of high frequency AC energy to tissue can heat the tissue through high frequency induced intracellular oscillation of ionized molecules, resulting in temperature elevation in the tissue. Cell death can occur in the tissue, for example, at about sixty degrees Celsius. At a range of about 60 to 99 degrees Celsius, coagulation of protein in the tissue can occur. When the temperature is above about boiling, vaporization of tissue cells can occur. Vaporization can be used to cut tissue.

### OVERVIEW

The present disclosure provides methods for coating an insulative component of a surgical device, and a coated insulation element of a surgical device. The surgical device can be, for example, a J-hook type RF energy cutting device, including an insulation element, such as a dielectric, in the tip of the device between electrodes.

In some cases, the insulation element can be subject to build-up during activation. This kind of build-up of proteins, tissue, or other components, can cause shorting or other breakdowns of the insulation element. For example, where the insulation element is ceramic, it can run away when build-up occurs; where a silicone rubber material is used, ash can form from carbon build-up and result in tracking.

Proposed herein in the use of a non-stick, hydrophobic layer, on the insulation element. The layer can be thin, and can be made of a material such as polysiloxanes or fluorosilanes. The layer can also help prevent sticking of tissue to the device. In some cases, the layer can include three dimensional structures. In some cases, the layer can include a coating.

Such as non-stick coatings and surface structures can be used with medical devices that may exhibit tissue buildup during medical procedures, and that heat up during operation. In some cases, when such medical devices heat up with tissue on the device, the carbonization of the tissue can occur and create these undesirable short circuit-based issues. For example, such non-stick layers can be used on J-hook or spatula type devices.

Non-stick coatings and surface structures can provide hydrophobic properties to such insulative elements, and prevent build-up of carbon or tissue on the surgical devices. This can aid in preventing shorting or flash outs of these devices.

In an example, a surgical device can include a longitudinal shaft having a distal portion and a proximal portion, the shaft for at least partial insertion into a patient; an end effector on the distal portion of the longitudinal shaft, wherein the end effector is configured to cut tissue, the end effector comprising at least one electrode, and an insulation element; and a non-stick layer at least partially covering the insulation element, wherein the non-stick layer comprises a material having a surface adherence to tissue that is less than a surface adherence to tissue of the material of the insulation element.

In an example, a method can include applying a non-stick layer to an insulation element of an end effector in a surgical device, wherein the non-stick layer comprises a hydrophobic surface.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram of an electrosurgical system including a surgical device with a coated insulation element in an example.

FIG. 2A is a schematic diagram of a distal portion of a J-hook surgical device with a coated insulation element in an example.

FIG. 2B is a schematic diagram of a distal portion of a spatula surgical device with a coated insulation element in an example.

FIG. 2C is a schematic diagram of a surface on a surgical device coating with a hydrophobic coating in an example.

FIG. 3 shows a surface including a hydrophobic physical structure in accordance with some example embodiments.

FIG. 4 shows another surface including a hydrophobic physical structure in accordance with some example embodiments.

FIG. 5 shows another surface including a hydrophobic physical structure in accordance with some example embodiments.

FIG. 6 is a flow chart depicting a method of applying a hydrophobic coating to a surgical device.

### DETAILED DESCRIPTION

The present disclosure describes, among other things, hydrophobic surfaces or layers on insulative elements of surgical devices such as J-hook and spatula electrode devices. For example, polysiloxane or fluorosilane layers can be used on insulative elements on such devices to prevent build-up of tissue during activation of the device. Tissue build up during electrode activation can sometimes result in shorting or other breakdowns of the insulative element. Such insulative elements can, for example, include ceramic or silicone materials. Similarly, hydrophobic coatings or structures can be applied to various contact areas to prevent sticking of tissue to the surface of the electrode.

Such non-stick coatings can therefore be used for cutting devices, such as on elements of those cutting devices made of insulative materials such as ceramic and silicon. If tissue build up occurs on those devices, it can become problematic because if the material heats up significantly and carbonizes (e.g., the insulative material is converted into carbon or charcoal, such as by heating or burning or during fossilization). A build of hot carbon on such a silicone material can produce the carbonization effect. Hot carbon is electrically conductive, so build-up of carbon and carbonization can cause short circuits and flare outs in the device. Using a non-stick layer, coating, or surface structure, such as polysiloxanes or fluorosilanes can potentially reduce short circuits and flare outs.

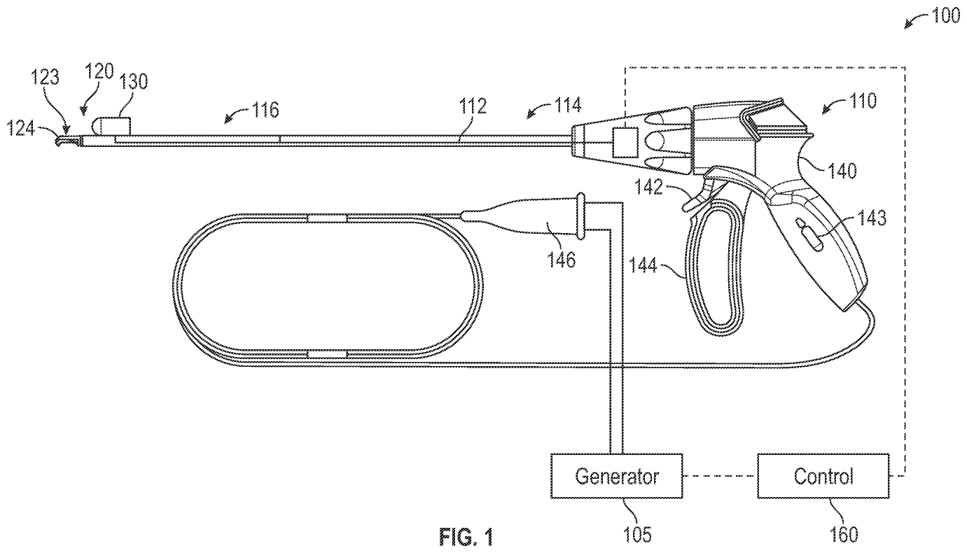

FIG. 1 illustrates a schematic diagram of an example of portions of an electrosurgery system 100, such as can include an electrosurgery device 110 with an electrosurgical end effector 120 such as a J-hook or spatula using RF energy. The device 110 can be connected to an electrosurgical energy generator 105 and a controller 160.

The electrosurgery device 110 can include an longitudinal shaft 112 having a proximal portion 114 and a distal portion 116. The distal portion 116 can include an end effector 120, such as which can include an insulation element 123, electrodes 124, and a non-stick layer 125. A proximal portion 114 of the device can be connected to a handpiece 140, such as with actuators 142, 143, and 144. The device 110 can also include a connector 146 such as can be configured to be connected to the generator 105.

The generator 105 can be external to but coupled to the electrosurgical device 110. The generator 105 can provide electrical energy to the end effector 120 of the electrosurgical device 110, such as through the electrical connector 146. The electrical generator 105 can produce a current deliverable by the end effector 120 such as for inducing a coagulation mode of electrosurgery. The electrical generator 105 can be in communication with the controller 160, which can direct the application of electrosurgical energy to the end effector 120 in the electrosurgical device 110.

The type and amount of electrical energy provided by the generator 105 can vary, such as depending on the desired treatment. The electrosurgical waveform produced, the voltage, and the power of the electrosurgical energy being delivered, and the size and surface area of the end effector 120, can affect the depth and the rate of producing heat, which, in turn, can alter the final effect on the target tissue.

The electrosurgery device 110 can include a bipolar electrosurgery end effector 120 such as for applying high-frequency alternating polarity electrical current to biological tissue, such as to cut, coagulate, desiccate, or fulgurate the tissue, such as may be desired by the surgeon treating the patient.

The electrosurgery device 110 can include a wet field device such as for wet field electrosurgery, such as in a saline solution, or in an open wound. In a wet field device, heating can result from an AC current passing between two electrodes. Heating can be the greatest where the current density is the highest. Thus, smaller surface area electrode can produce a greater amount of heat for treating tissue.

In the device 110, the shaft 112 with the proximal portion 114 and the distal portion 116 can be sized, shaped, or arranged for partial insertion of the device 110 into a patient. The shaft 112 can include or can be made of one or more of a composite, plastic, or metallic material, or other material suitable for surgical applications. The proximal portion 114 can be near an operator, such as a surgeon, when the device 110 is in use. In some cases, the operator can be a robotic arm or other machine. The distal portion 116 can be sized, shaped, or arranged for insertion into the patient so that distal portion 116 is further from the operator during use.

In some cases, the shaft 112 can be sized, shaped, arranged, or otherwise configured for laparoscopy, in some cases, the shaft 112 can be shorter such as for open surgery applications. In some cases, such as for laparoscopy, the shaft can be long. In an open surgery application, the shaft can include a tissue interface element with cutting, coagulating, and sensing elements in or on a distal portion of that device.

Laparoscopy can include, for example, a surgical procedure in which a small incision is made, through which a device is inserted to diagnose or treat conditions. Laparoscopy is considered less invasive than regular open abdominal surgery. In the case of laparoscopy, an optical visualization or imaging device may also be inserted along with the device 110, such as to permit the optical device to allow viewing or imaging such as for the operator to observe the tissue. The optical visualization or imaging device can include a laparoscope, or viewing tube, such as with a camera. In some cases, the optical visualization or imaging device can include an ultrasound type imaging device for the operator to use during treatment.

By contrast, open surgery approaches can involve a larger incision, such as can allow more direct visual observation of cutting of skin and tissue, such to permit the surgeon to have a fuller view of the structures and organs involved in the procedure.

For example, in some applications, the shaft 112 can have a length in a range of 10 mm to 30 mm, inclusive. The shaft 112 can be narrow in a cross-section or a lateral dimension, such as for patient insertion via an incision. For example, the shaft 112 can have a cross-sectional or lateral width in a range of less than 6 mm, inclusive.

The end effector 120 can be located at or near the distal portion 116 of the shaft 112. The end effector 120 can include a bipolar electrode such as for use in coagulating tissue. Bipolar electrodes can make use of high frequency electrical current such as to cut, coagulate, desiccate, or fulgurate tissue. With a bipolar electrode configuration, current passes through the tissue between two more closely-spaced electrodes, such as between individual electrode arms of a forceps-type electrode. In a bipolar configuration, the current passes through the tissue between tips of two active electrodes, such as between electrode tips of a bipolar forceps. The electrical generator 105 can be connected to both active and return electrodes, such as for sending and receiving current. The end effector 120 can be configured to heat the targeted tissue.

Tissue can see a reduction in resistance as it heats (assuming the fluid content remains unaltered and does not change to the more resistive state of steam). Therefore, during a procedure the risk increases of moving into the undesirable lower resistance range during the heating of some tissues. By increasing the resistance of the devices by just a few ohms (e.g., 5-10 Ohms), it can have a large impact on a device when connected to a limited source current generator, by essentially moving the power curve towards the lower resistance states.

Thus, by providing a device with a thin non-stick coating such as layer 125, the resistance of the device can be increased by an amount such that the benefit of having the ability for a lower current system to boil the material between the jaws at lower material resistances. In an example, settings of the generator will also have to be adjusted to accommodate this change for things such as short circuits and absolute detection points. For example, the artificial increase in the seen resistance, requires the value of short circuits to be raised by approximately the same amount as the artificial offset. Also, if other actual values are being monitored, for such things as a drying endpoint or boiling detection, the value also has to be offset to accommodate for this difference.

The handpiece 140 can include one or more user-actuators, such as the actuators, 143, 144. In some cases, these can include one or more of levers, buttons, wheels, switches, triggers, or a combination thereof. One of the actuators 142, 143, 144, can provide a user-interface to control a first switch that selectively connects the end effector 120 to the generator 105 or other circuitry that can provide electrosurgical energy to the first end effector 120 such as for cutting and coagulation. Additional actuators, such as buttons, triggers, or other user-actuatable mechanisms can be included on the handpiece 140 of the device 110 or elsewhere for surgeon use, such as for direction and action of the end effector 120, movement of the shaft 112, or one or more other operations of the device 110.

The electrosurgical device 110, including the triggers on the handpiece 140, the end effector 120, and the one or more sensors 130, can be in communication with the controller 160. The generator 105 can also be in communication with the controller 160.

The controller 160 can include a processor and a memory such as to permit the controller 160 to communicate with and control the generator 105. The controller 160 can be used to allow for both predictive and reactive control of the duty cycle produced by the generator 105.

The controller 160 can operate as a standalone device, or may be networked to other machines. The controller 160 can include a hardware processor, such as a central processing unit (CPU), a graphics processing unit (GPU), a hardware processor core, or any combinations thereof. The controller 160 can further include a memory, including a main memory and a static memory. The controller 160 can include an input device, such as a keyboard, a user interface, and a navigation device such as a mouse or touchscreen.

The controller 160 can additionally include a storage device, a signal generation device, a network interface device, and one or more sensors. The storage device can include a machine readable medium on which is stored one or more sets of data structure or instructions embodying or utilized by any one or more of the techniques described herein. The instructions may also reside, completely or at least partially, within the main memory, within static memory, or within the hardware processor during execution thereof by the controller.

In an example, one or any combination of the hardware processor, the main memory, the static memory, or the storage device may constitute machine readable media, that may include any medium that is capable of storing, encoding, or carrying instructions for execution by the controller 160 and that cause the controller 160 to perform any one or more of the techniques of the present disclosure, or that is capable of storing, encoding or carrying data structures used by or associated with such instructions. Non-limiting machine readable medium examples may include solid-state memories, and optical and magnetic media. The instructions on the controller 160 may further be transmitted or received over a communications network using a transmission medium via a network interface device.

The device 100 in FIG. 1 can be used, for example, to cut tissue, such as with RF (radio frequency) energy derived from the current produced by the generator. For cutting tissue, the generator 105 can produce an electrosurgical energy waveform similar to a sine wave. Cutting can use a continuous electrosurgical energy waveform such as can be able to apply the maximum output power of the generator 105, if desired. By comparison, for coagulating tissue, the peak current output can be greater than for cutting tissue, but with an intermittent or lower duty cycle waveform with lower average power than a cutting waveform. For cutting tissue, the peak voltage can be greater.

Cutting tissue can include resection and dissection. Various electrosurgical waveforms can be used for electrosurgical procedures. Rapid heating of tissue using a continuous waveform can result in vaporization, fragmentation, and ejection of tissue fragments, allowing for tissue cutting. Open circuit voltage of such electrical waveforms can be, for example, from about 300 to about 10,000 V peak-to-peak, inclusive. In some cases, rapid tissue heating can allow for explosive vaporization of interstitial fluid; if the voltage is sufficiently high, such as above 400 V peak-to-peak, the vapor can be ionized, sometimes resulting in conductive plasma allowing flow of electric current from the electrode via the plasma into the tissue.

Shown in FIG. 1, the end effector 120 can be a surgical cutting device, such as a J-hook type bipolar device, or other RF type cutting devices. In other cases, the device can be a spatula or other bipolar cutting instrument having an insulation element 123 on or near the cutting end of the device. The insulation element 123 can electrically separate and isolate different electrodes 124 from each other to prevent shorting therebetween in a bipolar configuration.

However, during operation, build-up of tissue on the insulation element can sometimes cause carbonization (e.g., turning to charcoal or carbon due to heating) of the insulation element 123. For example, as the end effector 120 is using RF energy to cut tissue, the insulation element 123 heats, and more carbon may build up on and stick to the surface of the insulation element 123. This can result in shorting between the two electrodes 124, which are no longer separated by an insulator. This can additionally produce flash outs, interrupting cutting work done with the electrodes 124. For example, where the insulative element is silicone or silicone rubber, free carbon materials during operation can “track” across the surface of the insulation element 123. This can cause carbon tracking across the surface of the insulation element 123. In this case, the build-up across the surface of the insulation element 123 can increase the temperature across the end effector 120 can potentially cause the silicone rubber to turn to ash (e.g., carbonize to charcoal), resulting in electrical shorting between the electrodes 124. Similarly, if the insulation element 123 is made of a ceramic, such build-up on the surface of the ceramic could cause melting of a portion of the device, damaging the insulative material and allowing shorting between the electrode 124.

Thus, the end effector 120 can further include a non-stick layer 125. The non-stick layer 125 can at least partially cover the insulation element 123. The non-stick layer 125 can have a surface adherence less than that of the insulation element 123. In some cases, the non-stick layer 125 can be a coating, such as polydimethylsiloxane, hexadimethylsiloxane, or tetramethyldisiloxane. In an example, the non-stick layer 125 can have a thickness in range of about 10 nm to about 300 nm. In some cases, the non-stick layer 125 can have a substantially uniform thickness. In some cases, the non-stick layer 125 can have a non-uniform thickness. In some cases, the non-stick layer 125 can be discontinuous. In some cases, the non-stick layer can be continuous. The non-stick layer 125 can include one or more asperities, such as nanoparticles. The non-stick layer 125 can include an electrically insulated or a non-conductive material. In some cases, the non-stick layer 125 can include a hydrophobic surface structure, a coating, or a combination thereof. In some cases, the non-stick layer 125 can overlap a portion of the electrode 124.

This layer 125 can be chosen and applied to the insulation element 123 of the end effector 120 to prevent or reduce sticking of tissue to the insulation element 123 of the device 110 and prevent carbonization of the insulative material. The non-stick layer 125 can, for example, include a nanostructure and a non-stick structure to reduce sticking of the coagulum to the tip of the device 110. The non-stick layer 125 can, for example, have a low surface energy, such as to prevent sticking of coagulum to the device. The non-stick layer 125 can allow for reduction of thermal transfer between the insulation element 123 and the target tissue, so as to reduce sticking therebetween. In some cases, the non-stick layer 125 can include super hydrophobic materials.

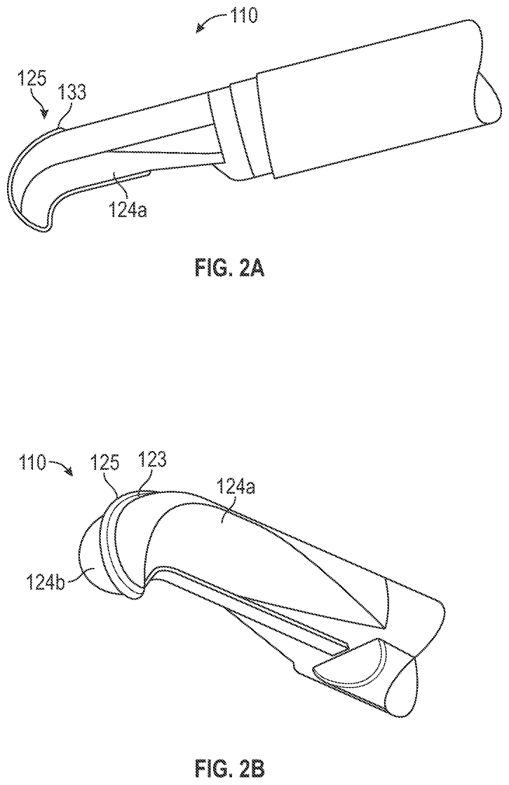

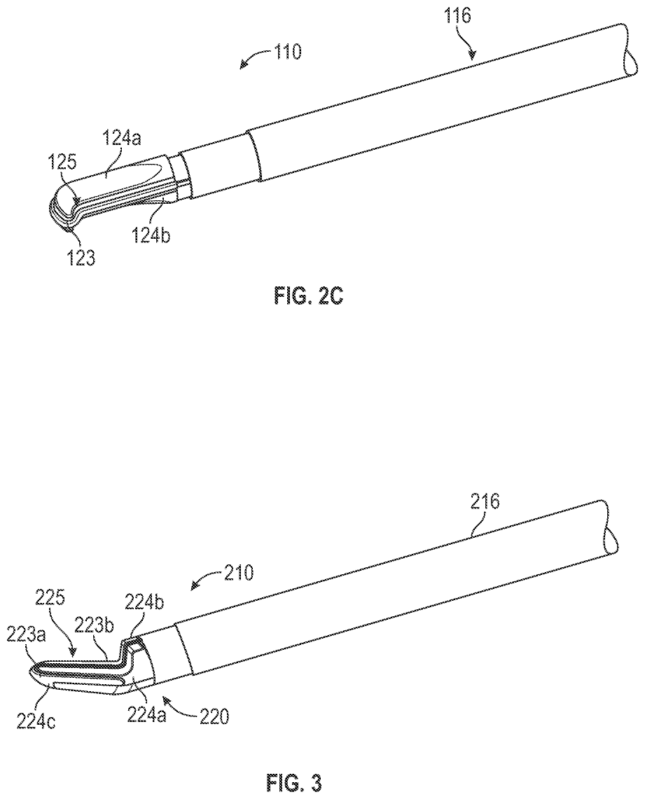

An example J-hook end effector 120 is shown in greater detail in FIGS. 2A-2C. The end effector 120 can extend distally from the shaft. The end effector 120 can include an insulation element 123 and electrode material 124, along with layer 125. The end effector 120 can be used, for example, for surgery such as colon surgery or intestinal surgery. The end effector 120 can be used for cutting tissue. The end effector 120 can further include the non-stick layer 125.

The insulation element 123 can be any suitable ceramic material or silicone material for hosting the electrodes 124 and insulting the electrodes 124 from each other. The electrode material 124 can serve as the electrical conductors along the probe tip 110. The electrode material 124 can be printed, etched, or adhered to the ceramic probe tip 122.

In an example, the layer 125 can include a non-electrically conductive coating on an external surface of the end effector 120, or an insulative coating. The layer 125 can have a resistance or impedance of less than about 10 ohms, or less than about 5 ohms. The layer 125 can have a surface adherence less than that of the electrode material 124. The layer 125 can, for example, have a coefficient of friction that is lower than that of the electrode material 124.

In some cases, the coating can be uniform in coverage and thickness, in some cases it can be fully or partially coating the end effector 120. The layer 125 can have a thickness of up to about 300 nm, up to about 200 nm, up to about 100 nm, or less. In some cases, the layer 125 can be hydrophobic or superhydrophic. In some cases, the layer 125 can have a nanostructure or microstructure to reduce stickiness.

Examples of the present disclosure provide for disposing a non-stick coating on components of an electrosurgical device at a particular thickness or within a particular range of thicknesses such that the non-stick coating provides adequate tissue sticking reduction during tissue sealing without negatively impacting tissue sealing performance of the vessel sealing instrument.

As discussed herein, a thickness of the non-stick coating can be in the range of 10 nm to about 300 nm and provide non-stick benefits. However, while non-stick properties can be provided, various portions of this range can provide additional benefits, while still providing tissue adhesion resistance and sensing capability. In one example, the non-stick coating can be a thin coating, e.g. having a thickness in the range of, but not limited to, about 10 nm to about 30 nm. In one example, the non-stick coating has a thickness in the range of about 10 nm to about 20 nm. In one example, the non-stick coating has a thickness less than 20 nm such as about 15 nm.

In one example, the non-stick coating has a predetermined number of activations and can promotes single-use. That is, the electrosurgical device or a portion of the electrosurgical device (including the jaw members) has a non-stick coating thickness that allows a surgeon to perform a particular procedure on a patient, but the non-stick properties reduce after the predetermined number of activations to discourage a user from attempting to sterilize and reuse the electrosurgical device (or a portion of the electrosurgical device) on a subsequent patient. Promoting single-use lessens the risk of cross-contamination and hospital acquired diseases.

In some cases, the non-stick coating thickness can be controlled to such that natural wearing of the non-stick coating that occurs within the number of predetermined activations and still provides non-stick properties. However, after the number of predetermined activations, the non-stick properties are reduced such that might be dissuaded from using the device for another full procedure. As discussed herein, the non-stick coating provides enhanced non-stick properties compared to an uncoated device. Once the non-stick coating wears, it can generally have similar anti-stick properties of the uncoated device.

For example, the number of predetermined activations can be from about 20 activations to about 50 activations. Additionally, non-stick properties can be provided by the non-stick coating for the predetermined number of activations. In one example, the non-stick coating has a thickness of about 15 nm and can be used to provide approximately 30 activations of enhanced anti-stick properties as compared to an uncoated device during 30 activations.

The non-stick layer 125 can allow passage of energy, such as RF energy, through such that the end effector 120 can affect the target tissue. For example, the layer 125 can be a light capacitive element or light resistive element, that allows passage of electrode energy through the layer 125. The layer 125 can be directly applied to the ceramic probe tip 122 and the electrode material 124. In some cases, an adhesive can be used to apply the layer 125 to the device 110.

As discussed herein, the non-stick coating can be applied to portions of the electrosurgical device to provide tissue adherence resistant (anti-stick) properties. Any material capable of providing the desired functionality (namely, reduction of tissue sticking while simultaneously maintaining sufficient electrical transmission to permit tissue sealing) may be used as the non-stick coating, provided it has adequate biocompatibility. In some examples, the material may be porous to allow for electrical transmission.

In some cases, the layer 125 can include a polymeric-based coating, such as a fluoropolymer type coating. In some cases, the layer 125 can include a Polytetrafluoroethylene (PTFE) coating. In some cases, the layer 125 can include a polysiloxane or a fluorosilane coating. For example, materials such as silicone and silicone resins can be used for the non-stick coating. In one example, the silicone and silicone resins can be applied using a plasma deposition process to precisely control thickness, and can withstand the heat generated during tissue sealing. Silicone resins suitable for the non-stick coating include, but are not limited to, polydimethyl siloxanes, polyester-modified methylphenyl polysiloxanes, such as polymethylsilane and polymethylsiloxane, and hydroxyl functional silicone resins. In some examples, the non-stick coating is made from a composition including a siloxane, which may include hexamethyldisiloxane, tetramethylsilane, hexamethyldisilazane, or combinations thereof

In an example, the non-stick coating is a polydimethylsiloxane (“PMDSO” coating. In one example, the non-stick coating is a hexamethyldisiloxane (“HMDSO”) coating. In another example, the non-stick coating is a tetramethyldisiloxane (TMDSO or TMDS). In some cases, the layer 125 can include a thin layer of hexamethyldisiloxane (HMDSO), of a thickness of a few nano meters. HMDSO is electrically resistive, but the thinness of the coating can allow passage of RF energy therethrough.

The application of the non-stick coating may be accomplished using any system and process capable of precisely controlling the thickness of the coating. In some examples, HMDSO is deposited on the electrically conductive sealing plates using plasma enhanced chemical vapor deposition (PECVD) or other suitable methods such as atmospheric pressure plasma enhanced chemical vapor deposition (AP-PECVD). For example, the application of the polydimethylsiloxane coating may be accomplished using a system and process that includes a plasma device coupled to a power source, a source of liquid and/or gas ionizable media (e.g., oxygen), a pump, and a vacuum chamber. The power source may include any suitable components for delivering power or matching impedance to the plasma device. More particularly, the power source may be any radio frequency generator or other suitable power source capable of producing electrical power to ignite and sustain the ionizable media to generate a plasma effluent. Application of the coating is discussed in more detail below with reference to FIG. 7.

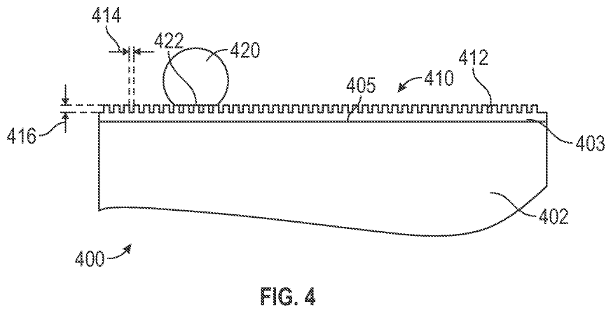

In some cases, the non-stick layer 125 can include an etched coating including one or more hydrophobic pillars superimposed on the electrode material 124. In some cases, the electrode material 124 itself can also be etched on the ceramic probe tip 122. With an etched layer 125, a nanostructure of hydrophobic pillars can act as a superhydrophobic coating with a low surface energy, reducing sticking. The etched layer 125 can be in any suitable pattern for the non-stick coating to reduce or prevent tissue sticking. The etched layer 125 can be applied, for example, by printing, chemical etching, laser etching, chemical bombardment, or other suitable techniques. Application of the coating is discussed in more detail below with reference to FIGS. 4-6.

In some cases, it may be beneficial to have different hydrophobic physical structures on different surfaces of components of the device. The hydrophobic physical structure may be on all or a portion of a surface of the device 110, and different hydrophobic physical structures may be used on different surfaces or components of a device. Example hydrophobic structures are discussed below in FIGS. 4-6.

FIGS. 2A-2C are views of a distal portion of a J-hook surgical device 110 with a coated insulation element in an example. The device 110 can include a distal portion 116 with the end effector 120. The end effector 120 can be J-shaped or hook shaped. The end effector 120 can include electrodes 124a, 124b, and insulation element 123. Non-stick layer 125 can be on top of insulation element 123.

The electrodes 124a, 124b, can allow for bipolar cutting of tissue with the end effector 120. Thus, each electrode 124a, 124b, can be set to an opposing polarity to allow flow of current from one electrode 124a, through tissue, and to the other electrode 124b. The electrodes 124a, 124b, can be separated by the insulation element 123. The insulation element 123 can be an electrically non-conductive material, such as a silicone rubber or a ceramic.

During tissue cutting, the generator 105 can send a current down to the electrodes 124a, 124b, which can allow application of RF energy at the point of contact between the J-hook end effector 120 and the targeted tissue. The insulation element 123 can prevent shorting of the bipolar J-hook.

However, during operation, free carbon may build up and stick to the insulation element 123. If free carbon builds up, for example on a siliconeinsulation element 123, the insulation element 123 can become carbonized. This can cause shorting between the two electrodes 124a, 124b. In the case of a ceramic electrode insulation element 123, this may cause ceramic material to run away. In some cases, cut tissue or coagulum may stick to the insulation element 123, blocking effective function of the insulation element.

For this reason, the non-stick layer is applied over the insulation element 123. The insulation element 123 can be a coating, physical hydrophobic structure, combinations thereof, or other appropriate non-stick surfaces. The non-stick layer 123 can help prevent buildup of free carbon and sticking of tissue to prevent such issues. This type of layer is discussed in more detail below with reference to FIGS. 4-6.

FIG. 3 is a schematic diagram of a distal portion of a spatula surgical device 210 with a coated insulation element in an example. The spatula surgical device 210 can be similar to the J-hook device discussed above, but the end effector 220 can have a different shape. The device 210 can be used for cutting tissue. The spatula surgical device 210 can include a distal portion 216 and end effector 220. The end effector can include electrodes 224a, 224b, 224c, and insulation elements 223a, 223b, with non-stick layer 225a, 225b. The end effector 220 can have a generally flatter, spatula type shape.

The spatula surgical device 210 can include two or more electrodes 224a, 224b, 224c, for use as a bipolar device for application of RF energy to tissue cutting. The electrodes 224a, 224b, 224c, can be separated by various insulation elements 223a, 223b, of which there can be more than one, to prevent shorting between various electrodes of differing polarities. The non-stick layer 225a, 225b, can be spread amongst the various insulation elements 224a, 224b, to prevent build-up and sticking of tissue or carbon, and prevent charring of insulation elements.

FIGS. 4-6 depict schematic diagrams of various examples of a non-stick layer that can be used on insulation elements of electrosurgery cutting device end effectors. FIG. 4 shows one example of a surface with a hydrophobic physical structure 310 on a substrate 302. As discussed in examples above, the hydrophobic physical structure 310 may be on all or a portion of a surface, and different hydrophobic physical structures 310 may be used on different surfaces or components of an electrosurgical cutting device. For example, the hydrophobic physical structure 310 may be on insulation elements of a cutting device end effector. The hydrophobic physical structure 310 may be on only a portion the end effector.

As shown in FIG. 4, in one example, the hydrophobic physical structure 310 includes asperities 312 having a height 316 and a pitch 314. The hydrophobic physical structure 310 can be described by the following equation:

$${Λ}_{c} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 )}$$

where Λ is a contact line density, and Λ_{c} is a critical contact line density; ρ=density of the liquid droplet; g=acceleration due to gravity; V=volume of the liquid droplet; θₐ=advancing apparent contact angle; θ_{a,0}=advancing contact angle of a smooth substrate; γ=surface tension of the liquid; and w=tower wall angle.

The contact line density Λ is defined as a total perimeter of asperities over a given unit area.

In one example, if Λ>Λ_{c} then a droplet 320 of liquid are suspended in a Cassie-Baxter state. Otherwise, the droplet 320 will collapse into a Wenzel state. In one example when a Cassie-Baxter state is formed, an ultra-hydrophobic condition exists, and a low adhesion surface is formed. FIG. 3 illustrates a Cassie-Baxter state, where the droplet 320 rests on top of the asperities 312 at interface 322. Although rectangular asperities are shown for illustration purposes, the invention is not so limited. Asperity shapes are taken into account in the formula above, at least in the tower wall angle (w) term.

In the example of FIG. 4, the asperities are formed directly from a bulk material, and are not formed from a separate coating. One method of forming asperities directly from a bulk material includes chemical etching. Another example of forming asperities directly from a bulk material includes laser etching or ablation. Another example of forming asperities directly from a bulk material includes ion etching.

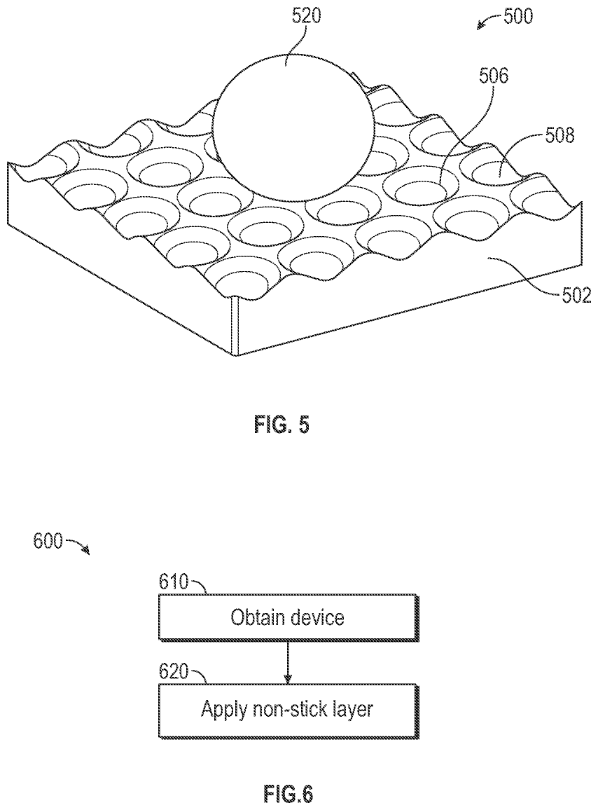

FIG. 5 shows another example of a surface with a hydrophobic physical structure 410 on a substrate 402. As discussed in examples above, the hydrophobic physical structure 410 may be on all or a portion of a surface, and different hydrophobic physical structures 410 may be used on different surfaces or components of an endoscope. For example, the hydrophobic physical structure 410 may be on an entire outer surface of an end effector. The hydrophobic physical structure 410 may be on only a portion of an outer surface of an end effector.

As shown in FIG. 5, in one example, the hydrophobic physical structure 410 includes asperities 412 having a height 416 and a pitch 414. However, in the example of FIG. 4, the hydrophobic physical structure 410 is formed as part of a coating 403 that forms a direct interface 405 with substrate 402. FIG. 5 illustrates a Cassie-Baxter state, where the droplet 420 rests on top of the asperities 412 at interface 422.

In one example, the asperities 412 are formed by application of nanoparticles to a surface of the substrate 402 to form the coating 403. In one example, the asperities 412 are formed by application of nanoparticles to a surface of the coating 403. In one example, the nanoparticles include hexamethyldisiloxane (HMDSO) particles. In one example, the nanoparticles include tetramethyldisiloxane (TMDSO) particles. In one example, the nanoparticles include fluorosilane particles. Other nanoparticle materials are also within the scope of the invention. In one example, a hydrophobic chemistry of the nanoparticle, in combination with a nano scale asperity structure as shown in FIG. 5 provide better hydrophobicity compared to a hydrophobic chemistry alone.

FIG. 6 shows one example of a laser etched surface 500 that includes hydrophobic physical structure as described above. In the example of FIG. 6, a gaussian hole array is formed by applying laser energy to a surface of a substrate 502 in a controlled regular pattern to form holes 506. A shape of the holes 506 is characterized as gaussian due to the energy distribution of laser energy in forming the array. In the example shown, a number of asperities 508 are formed in the process that may be spaced and arranged in an array that provides a Cassie-Baxter state as described above. A liquid droplet 520 is illustrated on the hydrophobic physical structure similar to the droplet 320 from FIG. 4, or the droplet 420 from FIG. 5.

FIG. 7 is a flow chart depicting a method 700 of applying a hydrophobic layer to a surgical cutting device.

The method 700 can include coating or etching the surface of a insulation elements of a bipolar cutting electrode, such as a J-hook or spatula, with a non-stick layer, such that the layer at least partially covers the insulation element. Application of the coating or layer can be done, for example, by chemical etching, laser etching, chemical bombardment, or printing.

In some cases, the coating can be produced in a uniform thickness of about 1 nm to about 300 nm, of about 5 nm to about 200 nm, or of about 10 nm to about 100 nm. In some cases, the coating can be produced in a pattern, such as to create hydrophobic pillars on the electrode. In some cases, the coating can fully or partially cover the electrode.

Several modification/application techniques may be used to form the coating, optionally including hydrophobic pillars. In one example, a sol-gel process can be used. Advantages of sol-gel application include the ability to coat more complex surfaces with high quality films. Challenges of sol-gel may include brittleness, limited thickness options, and induced mechanical stresses in the coating.

In one example, a cold spray process can be used. Advantages of cold spray application include the ability to coat at lower temperatures, with low deterioration, low oxidation, and low defects. Challenges of cold spray may include high energy needed for application, high cost, and a limited number of compatible substrates.

In one example, a chemical vapor deposition (CVD) process can be used. Advantages of CVD application include a high quality coating, high control of thickness, and the ability to coat complex surfaces. Challenges of CVD may include high temperature requirements, and high cost.

In one example, a physical vapor deposition (PVD) process can be used. Advantages of PVD application include the ability to coat inorganic compounds, ecological friendly processes, and a wide variety of available coating materials. Challenges of PVD may include high vacuum chamber requirements and high cost.

In one example, a thermal spray process can be used. Advantages of thermal spray application include a large selection of compatible coating materials and substrate materials, and low cost. Challenges of thermal spray may include difficulty in forming thick coatings, low adhesion issues of coatings, and ecologically unfriendly process steps.

In one example, an in-situ polymerization process can be used. Advantages of in-situ polymerization include the ability to coat with insoluble polymers. Challenges of in-situ polymerization may include process complexity, high cost, and limited potential for large scale production.

In one example, a spin coating process can be used. Advantages of spin coating include high quality coatings, fast drying times, and controllable thicknesses. Challenges of spin coating may include difficulty coating small surfaces and requirements of a smooth surface.

In one example, a dip coating process can be used. Advantages of dip coating include the ability to coat complex surfaces and the ability for large scale production. Challenges of dip coating may include undesirable solvent requirements, and limitations of only soluble polymer coatings.

In one example, an electrodeposition process can be used. Advantages of electrodeposition include high quality coatings at low cost. Challenges of electrodeposition may include long process times, and conductive substrate requirements.

Medical devices having a non-stick coating optionally including hydrophobic pillars as described show reduced adhesion over other non-textured coatings for bio materials including, but not limited to, tissues, blood, fats, and/or other biological materials. This provides clearer, less obstructed surfaces such as lenses for a number of possible medical devices, including, but not limited to, endoscopes. Application of hydrophobic physical structures to other surfaces of medical devices apart from optical components may further provide advantages such as reduced friction and reduced adhesion where desired.

### Various Notes & Examples

Example 1 can include a surgical device comprising a longitudinal shaft having a distal portion and a proximal portion, the shaft for at least partial insertion into a patient; an end effector on the distal portion of the longitudinal shaft, wherein the end effector is configured to cut tissue, the end effector comprising at least one electrode, and an insulation element; and a non-stick layer at least partially covering the insulation element, wherein the non-stick layer comprises a material having a surface adherence to tissue that is less than a surface adherence to tissue of the material of the insulation element.

Example 2 can include Example 1, wherein the non-stick layer comprises a coating selected from one of polydimethylsiloxane, hexadimethylsiloxane, and tetramethyldisiloxane.

Example 3 can include any of Examples 1-2, wherein the non-stick layer has a thickness within a range of about 10 nm to about 30 nm.

Example 4 can include any of Examples 1-3, wherein the non-stick layer has a substantially uniform thickness.

Example 5 can include any of Examples 1-4, wherein the non-stick layer is continuous.

Example 6 can include any of Examples 1-5, wherein the non-stick layer comprises hydrophobic pillars.

Example 7 can include any of Examples 1-6, wherein the non-stick layer comprises nanoparticles.

Example 8 can include any of Examples 1-7, wherein the non-stick layer comprises one or more asperities.

Example 9 can include any of Examples 1-8, wherein the non-stick layer comprises a non-conductive material.

Example 10 can include any of Examples 1-9, wherein the end effector comprises a plurality of insulation elements.

Example 11 can include any of Examples 1-10, wherein the non-stick layer comprises an electrically insulating material.

Example 12 can include any of Examples 1-11, wherein the non-stick layer comprises a hydrophobic surface structure, the hydrophobic surface structure having a lower surface adherence than the insulation element.

Example 13 can include any of Examples 1- 12, wherein the non-stick layer comprises a hydrophobic surface structure and a coating.

Example 14 can include any of Examples 1-13, wherein the non-stick layer overlays a portion of the at least one electrode.

Example 15 can include any of Examples 1-14, wherein the end effector comprises a J-hook or a spatula.

Example 16 can include a method comprising applying a non-stick layer to an insulation element of an end effector in a surgical device, wherein the non-stick layer comprises a hydrophobic surface.

Example 17 can include Example 16, wherein applying a non-stick layer comprises depositing a coating.

Example 18 can include any of Examples 16-17, wherein applying a non-stick layer comprises etching a hydrophobic surface structure.

Example 19 can include any of Examples 16-18, wherein depositing a non-stick layer comprises chemical vapor deposition on the insulation element.

Example 20 can include any of Examples 16-19, wherein depositing a non-stick layer comprises physical vapor deposition (PVD) on the insulation element.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A surgical device comprising:
a longitudinal shaft having a distal portion and a proximal portion, the shaft for at least partial insertion into a patient;
an end effector on the distal portion of the longitudinal shaft, wherein the end effector is configured to cut tissue, the end effector comprising at least one electrode, and an insulation element; and
a non-stick layer at least partially covering the insulation element, wherein the non-stick layer comprises a material having a surface adherence to tissue that is less than a surface adherence to tissue of the material of the insulation element.

2. The device of claim 1, wherein the non-stick layer comprises a coating selected from one of polydimethylsiloxane, hexadimethylsiloxane, and tetramethyldisiloxane.

3. The device of claim 1, wherein the non-stick layer has a thickness within a range of about 10 nm to about 300 nm.

4. The device of claim 1, wherein the non-stick layer has a substantially uniform thickness.

5. The device of claim 1, wherein the non-stick layer is continuous.

6. The device of claim 1, wherein the non-stick layer comprises hydrophobic pillars.

7. The device of claim 1, wherein the non-stick layer comprises nanoparticles.

8. The device of claim 1, wherein the non-stick layer comprises one or more asperities.

9. The device of claim 8, wherein the non-stick layer comprises a non-conductive material.

10. The device of claim 1, wherein the end effector comprises a plurality of insulation elements.

11. The device of claim 1, wherein the non-stick layer comprises an electrically insulating material.

12. The device of claim 1, wherein the non-stick layer comprises a hydrophobic surface structure, the hydrophobic surface structure having a lower surface adherence than the insulation element.

13. The device of claim 1, wherein the non-stick layer comprises a hydrophobic surface structure and a coating.

14. The device of claim 1, wherein the non-stick layer overlays a portion of the at least one electrode.

15. The device of claim 1, wherein the end effector comprises a J-hook or a spatula.

16. A method comprising:
applying a non-stick layer to an insulation element of an end effector in a surgical device, wherein the non-stick layer comprises a hydrophobic surface.

17. The method of claim 16, wherein applying a non-stick layer comprises depositing a coating.

18. The method of claim 16, wherein applying a non-stick layer comprises etching a hydrophobic surface structure.

19. The method of claim 16, wherein depositing a non-stick layer comprises chemical vapor deposition on the insulation element.

20. The method of claim 16, wherein depositing a non-stick layer comprises physical vapor deposition (PVD) on the insulation element.
